(19) United States
(12) Patent Application Publication
(10) Pub. No.: US 2025/0152044 A1
(43) Pub. Date: May 15, 2025

(54) WEARABLE METABOLIC SENSOR SYSTEMS

(71) Applicant: Cardiac Pacemakers, Inc., St Paul, MN (US)

(72) Inventors: Michael J. Kane, St Paul, MN (US); Yingbo Li, Woodbury, MN (US); Kenneth Martin Stein, Westhampton Beach, NY (US); Neal Patrick Peterson, Minneapolis, MN (US)

(21) Appl. No.: 18/774,681

(22) Filed: Jul. 16, 2024

Related U.S. Application Data
(60) Provisional application No. 63/548,008, filed on Nov. 10, 2023.

(51) Int. Cl. A61B 5/145; A61B 5/00; A61B 5/1473; A61B 10/00

(52) U.S. Cl. CPC A61B 5/14514; A61B 5/14546; A61B 5/1473; A61B 5/6833; A61B 2010/0006; A61B 2560/0252; A61B 2562/18

(57) ABSTRACT

Systems, methods, and devices feature a wearable device that includes a set of needles sized for access to interstitial fluid. The wearable device also includes a chemical indicator associated with each needle. A concentration of an analyte within the interstitial fluid can be determined based on one or more optical properties of the chemical indicator.

### CROSS REFERENCE TO RELATED APPLICATION

This application claims priority to Provisional Application No. 63/548,008, filed Nov. 10, 2023, which is herein incorporated by reference in its entirety.

### TECHNICAL FIELD

Instances of the present disclosure relate to wearable devices with analyte sensing technology.

### BACKGROUND

Analyte concentrations can help physicians determine appropriate treatment for patients with heart failure, chronic kidney disease, among other ailments.

### SUMMARY

In Example 1, a system includes a wearable device with a first set of needles sized for access to interstitial fluid and a first type of chemical indicator associated with each needle within the first set of needles.

In Example 2, the system of Example 1, the wearable device includes a second set of needles sized for access to interstitial fluid and a second type of chemical indicator associated with each needle within the second set of needles.

In Example 3, the system of Example 2, wherein the first type of chemical indicator includes a first material that changes color to indicate a first concentration of a first chemical in the interstitial fluid, wherein the second type of chemical indicator includes a second material that changes color to indicate a second concentration of a second chemical in the interstitial fluid.

In Example 4, the system of Example 3, wherein the wearable device further includes a third set of needles sized for access to interstitial fluid and a third type of chemical indicator associated with each needle within the third set of needles, wherein the third type of chemical indicator includes a third material that changes color to indicate a third concentration of a third chemical in the interstitial fluid.

In Example 5, the system of any of Examples 1-4, wherein the wearable device further includes a color reference.

In Example 6, the system of any of Examples 2-5, wherein the first type of chemical indicator is associated with sodium, wherein the second type of chemical indicator is associated with potassium.

In Example 7, the system of any of Examples 2-6, wherein the first type of chemical indicator and the second type of chemical indicator comprise a slurry or a film.

In Example 8, the system of any of Examples 2-7, wherein each needle in the first set of needles and in the second set of needles houses a respective diffusion membrane positioned distal to the respective first type of chemical indicators and second type of chemical indicators.

In Example 9, the system of any of Examples 1-8, wherein the wearable device further includes a moisture barrier comprising a window and arranged on the wearable device to seal the chemical indicators in the needles.

In Example 10, the system of Example 9, wherein the wearable device further includes a protective film removably coupled to the moisture barrier.

In Example 11, the system of any of Examples 1-10, wherein the wearable device further includes a seal removable coupled to the needles.

In Example 12, the system of any of Examples 1-11, wherein the wearable device further includes a temperature sensor.

In Example 13, the system of any of Examples 1-12, wherein the wearable device further includes a barcode.

In Example 14, the system of any of Examples 1-13, further including a smart phone comprising an image sensor. The smart phone is programmed to determine analyte concentrations based, at least in part, on a color of the chemical indicators.

In Example 15, the system of any of Examples 1-14, wherein the wearable device is a patch attachable to a patient's skin.

In Example 16, a system includes a wearable device with a first set of needles sized for access to interstitial fluid, a first type of chemical indicator associated with and positioned within each needle within the first set of needles, a second set of needles sized for access to interstitial fluid, and a second type of chemical indicator associated with and positioned within each needle within the second set of needles.

In Example 17, the system of Example 16, wherein the first type of chemical indicator includes a first material that changes color to indicate a first concentration of a first chemical in the interstitial fluid, wherein the second type of chemical indicator includes a second material that changes color to indicate a second concentration of a second chemical in the interstitial fluid.

In Example 18, the system of Example 17, wherein the wearable device further includes a third set of needles sized for access to interstitial fluid and a third type of chemical indicator associated with and positioned within each needle within the third set of needles.

In Example 19, the system of Example 18, wherein the third type of chemical indicator includes a third material that changes color to indicate a third concentration of a third chemical in the interstitial fluid.

In Example 20, the system of Example 16, wherein the wearable device further includes color references positioned throughout the wearable device adjacent to the first type of chemical indicators and the second type of chemical indicators.

In Example 21, the system of Example 16, wherein the first type of chemical indicator is associated with sodium, wherein the second type of chemical indicator is associated with potassium.

In Example 22, the system of Example 16, wherein the first type of chemical indicator and the second type of chemical indicator comprise a slurry or a film.

In Example 23, the system of Example 16, wherein each needle in the first set of needles and in the second set of needles houses a respective diffusion membrane positioned distal to the respective first type of chemical indicators and second type of chemical indicators.

In Example 24, the system of Example 16, wherein the wearable device further includes a moisture barrier comprising a window and arranged on the wearable device to seal the first type of chemical indicator in the first set of needles and to seal the second type of chemical indicator in the second set of needles.

In Example 25, the system of Example 24, wherein the wearable device further includes a protective film removably coupled to the moisture barrier.

In Example 26, the system of Example 25, wherein the wearable device further includes a seal removable coupled to the first set of needles and to the second set of needles.

In Example 27, the system of Example 16, wherein the wearable device further includes a temperature sensor.

In Example 28, the system of Example 27, wherein the temperature sensor changes color in response to temperature.

In Example 29, the system of Example 16, further including a smart phone comprising an image sensor. The smart phone is programmed to: (1) determine a first concentration of a first analyte based, at least in part, on a first color of the first type of chemical indicator, and (2) determine a second concentration of a second analyte based, at least in part, on a second color of the second type of chemical indicator.

In Example 30, the system of Example 16, wherein the wearable device is a patch attachable to a patient's skin.

In Example 31, a method includes capturing a digital image of a wearable device attached to a patient, where the wearable device includes a first set of chemical indicators and a second set of chemical indicators. The method further includes estimating a first analyte concentration based on a first color of the first set of chemical indicators; and estimating a second analyte concentration based on a second color of the second set of chemical indicators.

In Example 32, the method of Example 31, wherein the digital image includes color reference sections on the wearable device, wherein the estimating the first analyte concentration and the estimating the second analyte concentration are both further based on the color references.

In Example 33, the method of Example 32, wherein the wearable device includes a temperature sensor, wherein the estimating the first analyte concentration and the estimating the second analyte concentration are both further based on an output of the temperature sensor.

In Example 34, the method of Example 33, wherein the output is a color of the temperature sensor.

In Example 35, the method of Example 31, wherein the first analyte concentration is a sodium concentration, wherein the second analyte concentration is a potassium concentration.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIGS. 2-4 show side views of various portions of a chemical sensing device, in accordance with certain instances of the present disclosure.

FIGS. 5-7 show top views of different arrangements of a chemical sensing device, in accordance with certain instances of the present disclosure.

FIGS. 8-10 show block diagrams of methods for use with the chemical sensing system, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Analyte concentration measurements can help physicians determine treatment for patients with heart failure and chronic kidney disease, among other ailments. However, determining a patient's analyte concentrations can require drawing multiple blood samples from a patient at a clinic and processing the blood samples at a laboratory. Certain instances of the present disclosure are directed to systems, methods, and devices that feature wearable devices such as patches with analyte sensing technology.

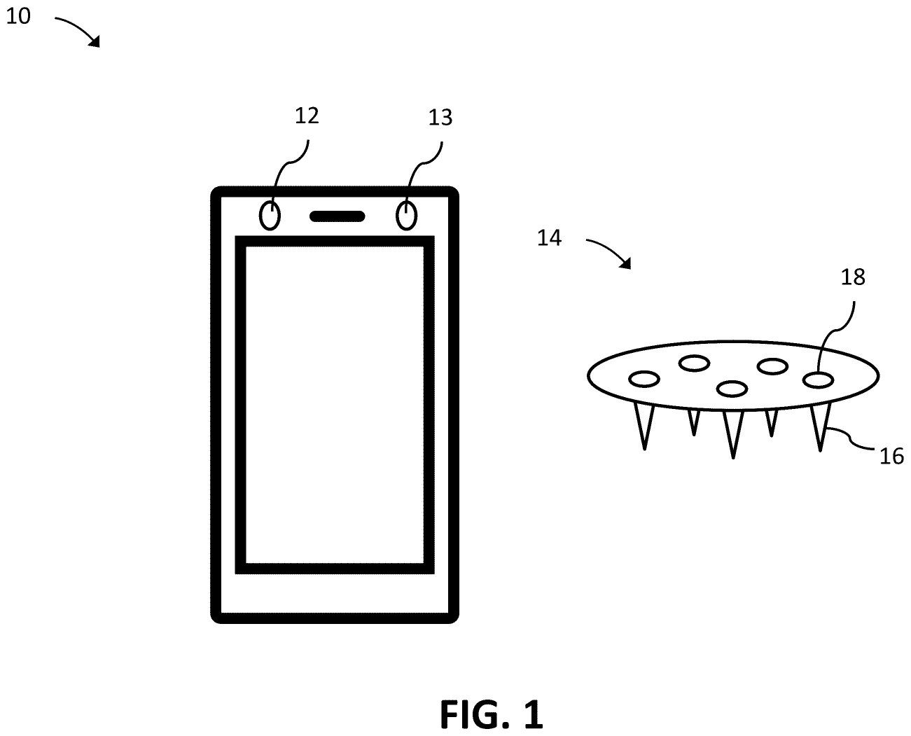

FIG. 1 shows a chemical sensing system 10 with schematic representations of a device with an image sensor 12 and a chemical sensing device 14.

The image sensor 12 (e.g., a charge coupled device, a complementary metal oxide semiconductor, or other devices that can capture an image) can be part of a camera, smart phone, or other device able to capture an image (e.g., a digital image). In certain instances, the image sensor 12 and the chemical sensing device 14 are integrated into a single device, and in other instances the image sensor 12 and the chemical sensing device 14 are separate devices. In instances where the image sensor 12 is part of a smart phone, the smart phone can store or otherwise access a program (e.g., a phone application) that processes an image (of the chemical sensing device 14) taken by the image sensor 12 and determines estimates of one or more analyte concentrations of the patient. In other instances, the image sensor 12 is part of a dedicated readout device or part of a camera. The system 10 can include one or more light sources 13, which can be part of the same device as the image sensor 12 or which can be part of a separate component. The one or more light sources 13 can generate light (e.g., emit visible light, ultraviolet light, monochromatic light (red, green, blue)).

The chemical sensing device 14 can be a wearable device (e.g., an exterior device and not an implantable device) such as a device that includes (or is part of) a strap (e.g., an armband strap), a patch (e.g., a torso patch), or another type of device that can be coupled to a patient's skin. For simplicity, the chemical sensing device 14 is hereinafter referred to as the “patch 14” although other types of wearable devices can use the chemical sensing technology described herein.

In certain instances, the patch 14 is a transdermal patch that includes multiple needles 16 (e.g., microneedles) sized to access a patient's interstitial fluid. The patch 14 can also include multiple chemical indicators 18, each of which changes color with changes in concentration of a certain analyte in the interstitial fluid. As described in more detail herein, the image sensor 12 can be used to capture an image (e.g., a digital image) of the chemical indicators 18, and the image can be processed and analyzed to determine respective concentrations of targeted analytes. In certain instances, the patch 14 includes one type of chemical indicator 18 (e.g., to help determine concentration of one type of analyte), but in other instances the patch 14 includes multiple types of chemical indicators.

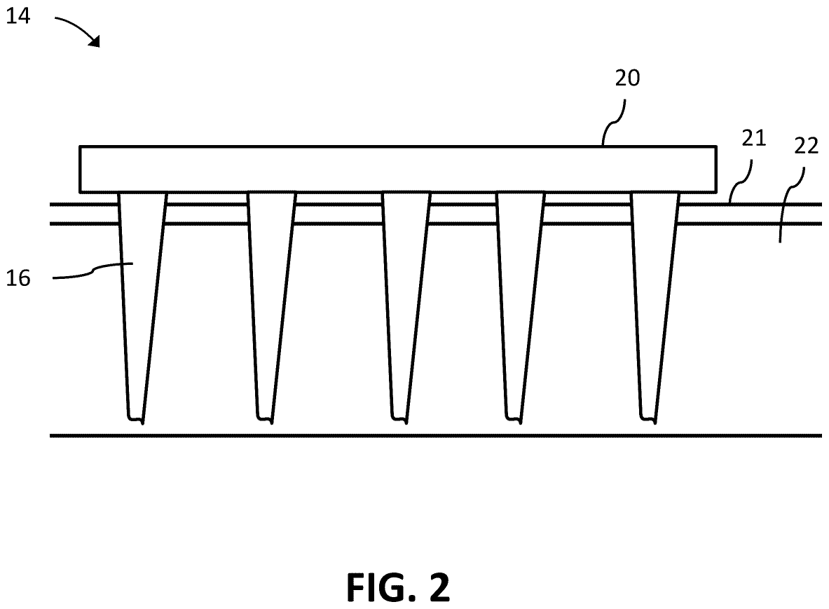

FIG. 2 shows a schematic side view of the patch 14 being worn by a patient. The patch 14 can be coupled to the patient's skin 20 such that the needles 16 pierce through the outer layer of skin 20 and extend into the patient's interstitial fluid space 22. The needles 16 can have openings that are exposed to the patient's interstitial fluid (and therefore analytes within the patient's interstitial fluid).

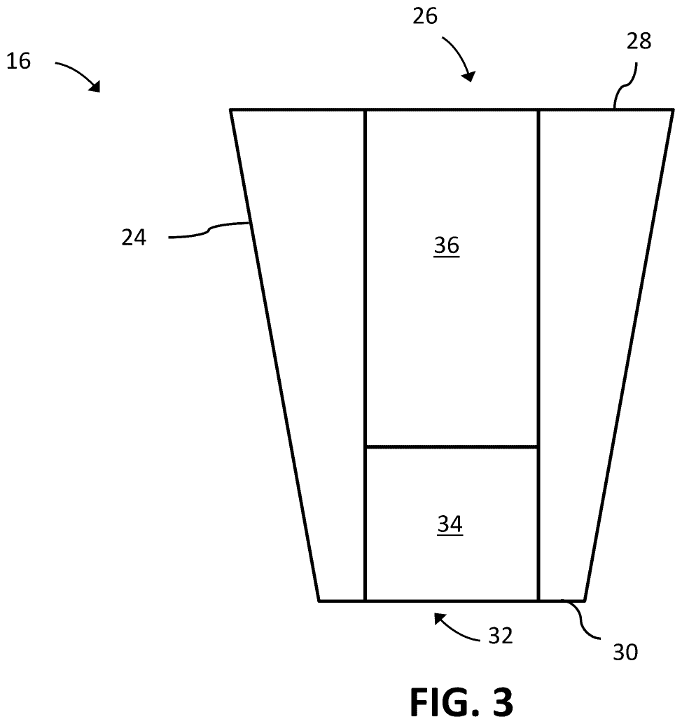

FIG. 3 shows a schematic side view of one of the needles 16 of the patch 14. In certain instances, the needles 16 are hollow needles such that each needle 16 includes an outer needle structure 24 that surrounds an opening 26 (e.g., a central thru-hole within the needle 16). The opening 26 can extend from a proximal end 28 of the needle 16 to a distal end 30 of the needle 16. An aperture 32 is located at or near the distal end 30 of the needle 16 such that the opening 26 is exposed to interstitial fluid.

Also at or near the distal end 30 of the needle 16 is a membrane 34 (e.g., a diffusion membrane) that is positioned within the needle 16. The membrane 34 protects tissue from direct interaction or exposure to a chemical indicator 36 that is also positioned within the needle 16. The membrane 34 can be formed from a permeable material, such as an ion permeable polymeric matrix material. In some instances, the membrane 34 can be permeable to sodium ions, potassium ions, hydronium ions, creatinine, urea, and various additional analytes. As referenced above, the cover membrane of the sensing element can be formed of a permeable material. In some embodiments, the cover membrane can be formed from an ion-permeable polymeric matrix material. Suitable polymers for use as the ion-permeable polymeric matrix material can include, but are not limited to, polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In certain instances, the membrane 34 includes an active agent disposed therein including, but not limited to anti-inflammatory agents, angiogenic agents, and the like.

The particular type (e.g., type of ion selectivity) and length of membrane can vary by needle 16. For example, one set of needles 16 can include a membrane 34 that is permeable to sodium ions, while another set of needles 16 includes a membrane 34 that is permeable to potassium ions, and so on. In other examples, the membrane 34 is agnostic to a particular type of ion. The membrane 34 is positioned such that analytes must pass through the membrane 34 before reaching the chemical indicator 36. The membrane 34 material used will affect how fast an analyte travels between interstitial fluid and the chemical indicator 36.

The chemical indicator 36 comprises a material that changes properties (e.g., optical properties such as color) with changes in concentration of a given analyte. In certain instances, color of the chemical indicator 36 comprises the sum of the absorption, transmission, reflectance, and fluorescence properties of the chemical indicator material. Put another way, the chemical indicator 36 can comprise a material that changes optical properties with changes in concentration of a given analyte- and such optical properties can be measured by analyzing an image of the chemical indicator 36.

The particular material of the chemical indicator 36 can vary by needle 16. For example, one set of needles 16 can include a chemical indicator 36 that responds to changes in sodium concentration, while another set of needles 16 includes a chemical indicator 36 that responds to changes in potassium concentration, and so on. In certain instances, the chemical indicator 36 has a minimum thickness or height along a longitudinal axis of a needle of 0.15-0.60 mm (e.g., 0.50-0.60 mm). In certain instances, the chemical indicator 36 comprises a slurry or a film.

In certain instances, the chemical indicator 36 is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 36 can include components (or response elements) that are configured for a colorimetric response, a photoluminescent response, or another optical sensing modality. For example, the chemical indicator 36 can include an element that changes color based on binding with or otherwise complexing with a specific chemical analyte. In some instances, the chemical indicator 36 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 36 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

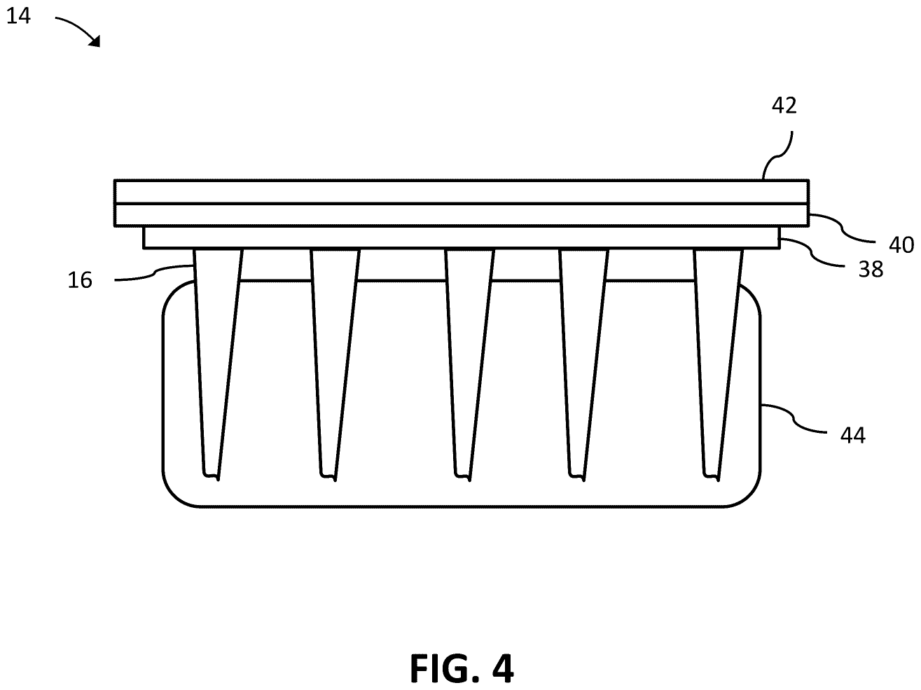

FIG. 4 shows a schematic side view of the patch 14 before being coupled to a patient (e.g., after manufacturing but before application to the patient). The needles 16 are coupled to and extend from a substrate 38. The substrate 38 can include respective openings or windows that couple to the openings 26 of the needles 16. As described in more detail below, the substrate 38 can include or be coupled to various color reference markers.

The patch 14 can also include a moisture barrier 40 that is coupled to the substrate 38. The moisture barrier 40 can include or form a window (e.g., a transparent window) such that optical properties (e.g., color) of the chemical indicators 36 (and color reference markers) can be viewed/observed from above the patch 14. The moisture barrier 40 can provide a vapor barrier such that the chemical indicators 36 (or components thereof) and analytes cannot pass through the moisture barrier 40. Put another way, the moisture barrier 40 can provide a seal that helps prevent liquid from leaking from the patch 14 such that the chemical indicators 36 remain positioned within the needles 16. The substrate 38 and the moisture barrier 40 can be flexible such that the patch 14 can be applied to curved parts of the patients and maintain contact when the patient moves.

The patch 14 can include a protective film 42 and a seal 44. The protective film 42 can be removably coupled to the moisture barrier 40 such that the protective film 42 can be removed right before or even after the patch 14 is applied to the patient. The seal 44 can be removably coupled to the needles 16 such that the needles 16 are not exposed during transit/shipping of the patch.

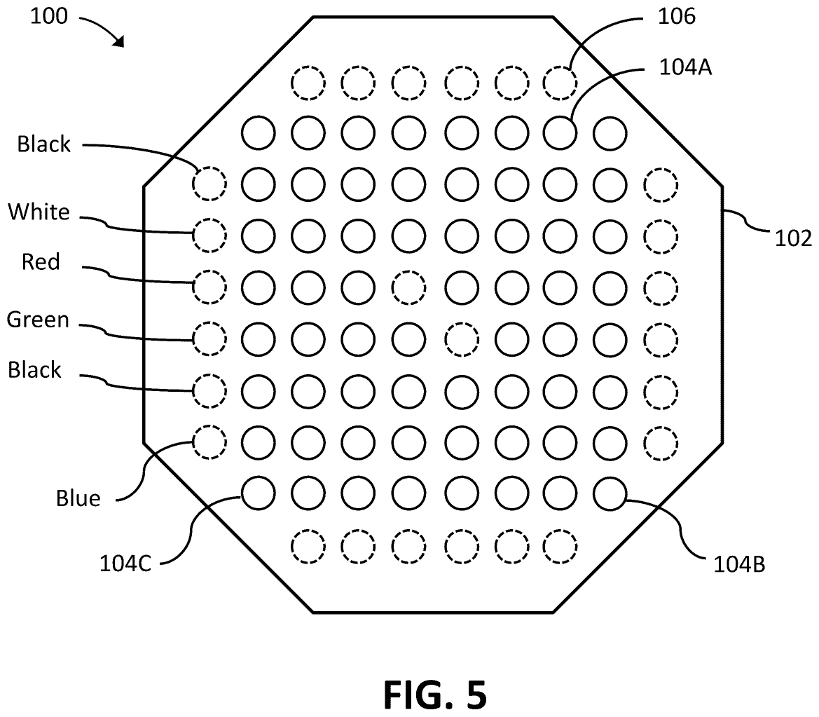

FIGS. 5-7 show top views of different arrangements of a patch. Each of the arrangements can be used in connections with the needles, membranes, chemical indicators, and layers, etc., described above with respect to the patch 14. The views shown in FIGS. 5-7 are the types of views of a patch that an image sensor would capture in a digital image while the patch is coupled to the patient. The digital image can capture the colors of the chemical indicators (and color references) such that the colors can be analyzed to determine concentrations of one or more analytes of the patient. The patch designs and features thereof in FIGS. 5-7 are not mutually exclusive and can be combined with each other to create other patch designs.

FIG. 5 shows a patch 100, which includes a window 102 through which various components of the patch 100 can be viewed. In particular, the window 102 allows chemical indicators 104A-C of the patch 100 to be viewed. The chemical indicators 104A-C can be positioned in needles (e.g., hollow needles) such as the needles described above with respect to FIGS. 1-4. As such, each chemical indicator 104A-C can be associated with its own needle.

A first set of needles can include a first type of chemical indicator 104A such as a chemical indicator that changes in color with changes in concentration of a first analyte (e.g., sodium). A second set of needles can include a second type of chemical indicator 104B such as a chemical indicator that changes in color with changes in concentration of a second analyte (e.g., potassium). A third set of needles can include a third type of chemical indicator 104C such as a chemical indicator that changes in color with changes in concentration of a third analyte (e.g., glucose). The respective colors of the chemical indicators can be used to estimate the respective concentrations of analytes in a patient's interstitial fluid.

In certain instances, each of the first type of chemical indicators 104A are positioned near or next to each other, each of the second type of chemical indicators 104B are positioned near or next to each other, and so on. The overall number of chemical indicators (and therefore the number of needles) and the number of different sets of types of chemical indicators on a given patch can be fewer or greater than that shown in FIG. 5. For example, the patch 100 could include a single type of chemical indicator selected for a single type of analyte. The relative positions of the chemical indicators 104A-C can vary from that shown in FIG. 5, and the specific shape of the chemical indicators 104A-C (as seen from a top view) can vary from the circular shapes shown in FIG. 5.

The patch 100 can also include color references 106. The color references 106 are shown in dotted lines in FIG. 5. The color references 106 can help with calibrating, correcting, and/or processing the digital image of the chemical indicators 104A-C such that an accurate estimate of the color of the chemical indicators 104A-C can be determined. For example, because the color of the color references 106 is known, the color of the chemical indicators 104A-C can be more accurately estimated as the patch 100 is positioned in different lighting (e.g., in direct sunlight, in a shadow, partially shaded, and the like). As such, the color references 106 can act as a color index or reference point for correcting for changes in color caused by ambient light.

In certain instances, some of the color references 106 are black, others white, others red, others green, others blue. Although most of the color references 106 in FIG. 5 are shown around a perimeter of the patch (e.g., with the chemical indicators 104A-C positioned within the perimeter), other positions and arrangements of the color references 106 can be utilized in the patch 100. The overall number and the specific shape of the color references 106 (as seen from a top view) can vary from the circular or dot shape shown in FIG. 5.

In certain instances, the patch 100 does not include any active electronics (e.g., does not include computing components or electronic sensors). In such instances, the patch 100 does not require batteries or another power source to function as designed.

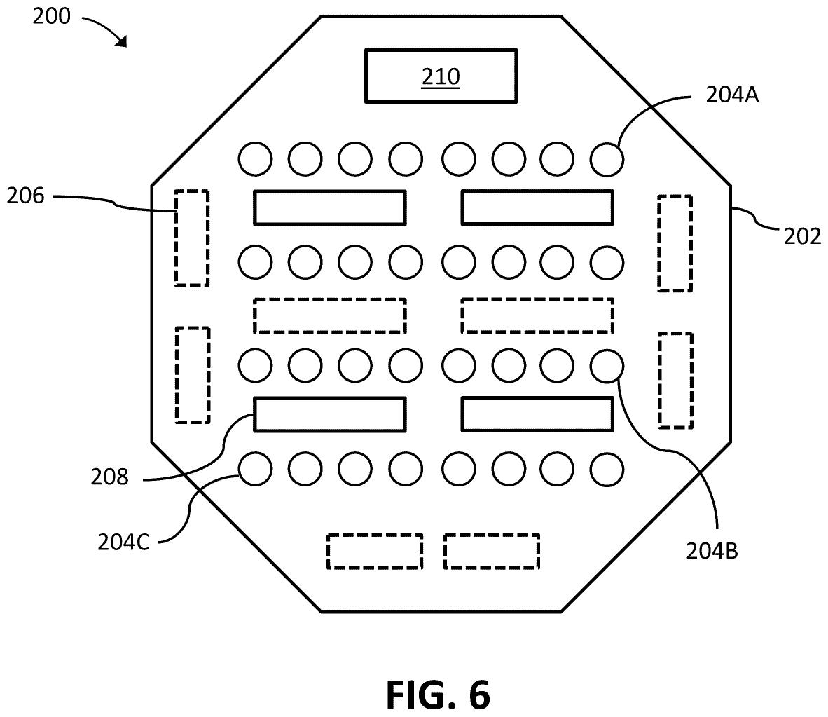

FIG. 6 shows a patch 200, which includes a window 202 through which various components of the patch 200 can be viewed. In particular, the window 202 allows chemical indicators 204A-C of the patch 200 to be viewed. The chemical indicators 204A-C can be positioned in needles (e.g., hollow needles) such as the needles described above with respect to FIGS. 1-4. As such, each chemical indicator 204A-C can be associated with its own needle.

A first set of needles can include a first type of chemical indicator 204A such as a chemical indicator that changes in color with changes in concentration of a first analyte (e.g., sodium). A second set of needles can include a second type of chemical indicator 204B such as a chemical indicator that changes in color with changes in concentration of a second analyte (e.g., potassium). A third set of needles can include a third type of chemical indicator 204C such as a chemical indicator that changes in color with changes in concentration of a third analyte (e.g., glucose). The respective colors of the chemical indicators can be used to estimate the respective concentrations of analytes in a patient's interstitial fluid.

In certain instances, each of the first type of chemical indicators 204A are positioned near or next to each other, each of the second type of chemical indicators 204B are positioned near or next to each other, and so on. The overall number of chemical indicators (and therefore the number of needles) and the number of different sets of types of chemical indicators on a given patch can be fewer or greater than that shown in FIG. 6. Further, the relative positions of the chemical indicators 204A-C can vary from that shown in FIG. 6. Further yet, the specific shape of the chemical indicators 204A-C (as seen from a top view) can vary from the circular shape shown in FIG. 6.

The patch 200 can also include color references 206. The color references 206 are shown in dotted lines in FIG. 6. In certain instances, some of the color references 206 are black, others white, others red, others green, others blue. The positions and arrangements of the color references 206 can vary from that shown in FIG. 6. The overall number and the specific shape of the color references 206 (as seen from a top view) can vary from the rectangular shape shown in FIG. 6.

The patch 200 can include one or more temperature sensors 208. The temperature sensors 208 can include a material (e.g., a liquid crystal material) that changes color with changes to temperature. Because the chemical indicators 204A-C may be sensitive to temperature, the color of the temperature sensors 208 can be used for calibrating, correcting, and/or processing the digital image of the chemical indicators 204A-C such that an accurate estimate of the color of the chemical indicators 204A-C can be determined.

The patch 200 can also include a bar code 210 (e.g., a one-dimensional bar code or a two-dimensional bar code). The bar code 210 can assist with identifying the patient, the model of the patch 200, the serial number of the patch 300, the arrangement of the patch 200, and/or the orientation 200 of the patch 200.

In certain instances, the patch 200 does not include any active electronics (e.g., does not include computing components or electronic sensors). In such instances, the patch 200 does not require batteries or another power source to function as designed.

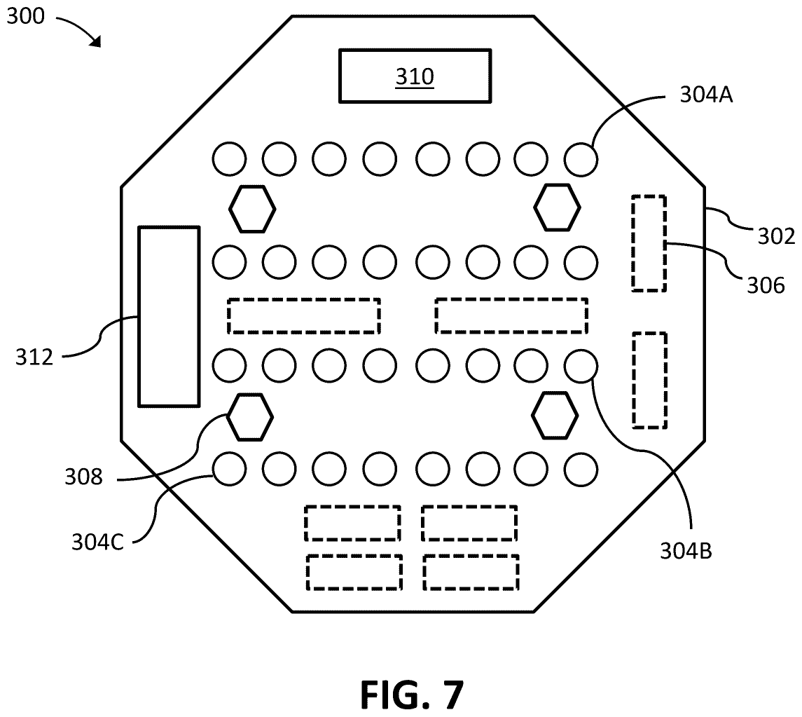

FIG. 7 shows a patch 300, which includes a window 302 through which various components of the patch 300 can be viewed. In particular, the window 302 allows chemical indicators 304A-C of the patch 300 to be viewed. The chemical indicators 304A-C can be positioned in needles (e.g., hollow needles) such as the needles described above with respect to FIGS. 1-4. As such, each chemical indicator 304A-C can be associated with its own needle.

A first set of needles can include a first type of chemical indicator 304A such as a chemical indicator that changes in color with changes in concentration of a first analyte (e.g., sodium). A second set of needles can include a second type of chemical indicator 304B such as a chemical indicator that changes in color with changes in concentration of a second analyte (e.g., potassium). A third set of needles can include a third type of chemical indicator 304C such as a chemical indicator that changes in color with changes in concentration of a third analyte (e.g., glucose). The respective colors of the chemical indicators can be used to estimate the respective concentrations of analytes in a patient's interstitial fluid.

In certain instances, each of the first type of chemical indicators 304A are positioned near or next to each other, each of the second type of chemical indicators 304B are positioned near or next to each other, and so on. The overall number of chemical indicators (and therefore the number of needles) and the number of different sets of types of chemical indicators on a given patch can be fewer or greater than that shown in FIG. 7. Further, the relative positions of the chemical indicators 304A-C can vary from that shown in FIG. 7. Further yet, the specific shape of the chemical indicators 304A-C (as seen from a top view) can vary from the circular shape shown in FIG. 7.

The patch 300 can also include color references 306. The color references 306 are shown in dotted lines in FIG. 7. In certain instances, some of the color references 306 are black, others white, others red, others green, others blue. The positions and arrangements of the color references 306 can vary from that shown in FIG. 7. The overall number and the specific shape of the color references 306 (as seen from a top view) can vary from the rectangular shape shown in FIG. 7.

The patch 300 can include one or more temperature sensors 308. The temperature sensors 308 can include sensors such as thermistors, thermocouples, or semiconductor junctions. Because the chemical indicators 304A-C may be sensitive to temperature, the temperature sensors 308 can be used for calibrating, correcting, and/or processing the digital image of the chemical indicators 304A-C such that an accurate estimate of the color of the chemical indicators 304A-C can be determined.

The patch 300 can also include a bar code 310. The bar code 310 can assist with identifying the patient, the model of the patch 300, the serial number of the patch 300, the arrangement of the patch 300, and/or the orientation 300 of the patch 300.

The patch 300 can also include a radio frequency identification (RFID) chip 312. In certain instances, the RFID chip 312 includes the temperature sensors 308. Further, the RFID chip 312 can store information such as identifying the patient, the model of the patch 300, the serial number of the patch 300, and the arrangement of the patch 300, etc. The RFID chip 312 can be designed to pair with certain reading devices that can access the information wirelessly.

In certain instances, the patch 300 does not include any active electronics (e.g., does not include computing components or electronic sensors). In such instances, the patch 300 does not require batteries or another power source to function as designed.

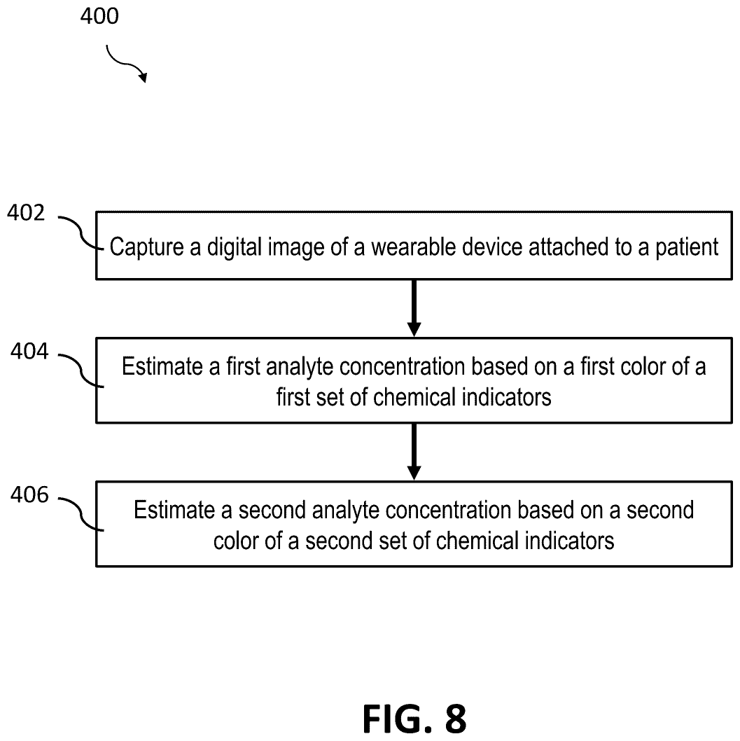

FIG. 8 shows a block diagram of a method 400 for estimating one or more analyte concentrations. The method 400 includes capturing a digital image of a wearable device attached to a patient (block 402). The method 400 further includes estimating a first analyte concentration based on a first color of the first set of chemical indicators (block 404). The method 400 further includes estimating a second analyte concentration based on a second color of the second set of chemical indicators (block 406). In certain instances, estimating the analyte concentrations involves calculating an analyte concentration for multiple chemical indicators and then applying a mathematical operation (e.g., averaging, voting) to determine the respective analyte concentrations. The analyte concentration estimations can be further based on corrections that are determined using color reference sections of the wearable device (e.g., a patch).

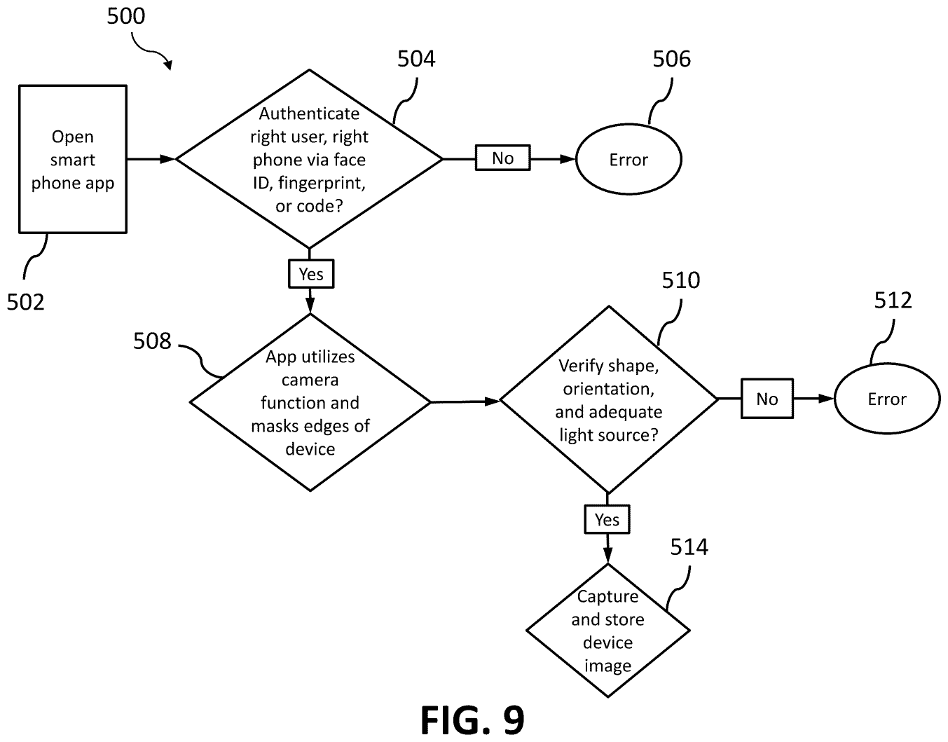

FIG. 9 shows a flowchart of a method 500 for capturing an image of a wearable device using an application on a smart phone. After opening the application (block 502) or even before the application is opened, the smart phone runs an authentication process to confirm that the correct patient is using the smart phone or application (block 504). The authentication process can involve checking items such as a fingerprint, facial recognition, a code or password, and/or the like. If the patient cannot be authenticated, the application determines that an error has occurred (block 506). Once authentication is confirmed, the application can access one or more cameras of the smart phone (block 508). In certain instances, the camera function of the smart phone can be used to determine and mask the edges of the wearable device. Next, the application can verify various aspects of the wearable device (block 510). This can involve verifying the shape of the wearable device, the orientation of the wearable device, and adequate light source. If certain aspects of the wearable device cannot be verified, the application determines that an error has occurred (block 512). Once the wearable device is verified, the one or more cameras can capture a digital image of the wearable device and store the digital image to local memory of the smart phone (block 514).

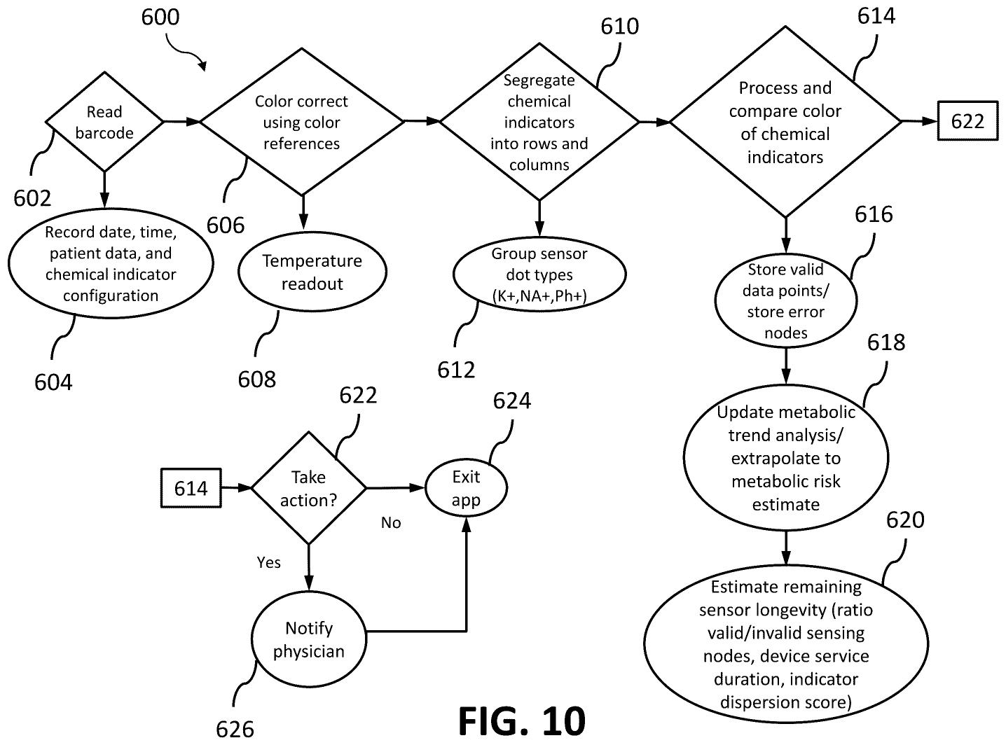

FIG. 10 shows a flowchart of a method 600 for determining estimated analyte concentrations based on an image captured by an image sensor. The method 600 can include reading a barcode (or other identifying indicator) from the digital image or otherwise accessing identifying information about a wearable device such as via an RFID chip (block 602). In response, the method 600 can include recording information (block 604) such as the date, time, patient data, and wearable data (e.g., arrangement of chemical indicators, type of chemical indicators).

The method 600 further includes correcting colors of the chemical indicators from the digital image using color references from the digital image (block 606). Further, the method 600 can include determining temperature (block 608) based on information in the digital image or via another process. If the wearable device includes multiple temperature sensors, the overall temperature can be determined by averaging the temperature measurements.

The chemical indicators in the digital image can be segregated into separate sets, with each set grouping together chemical indicators that are the same type (blocks 610 and 612). In certain instances, the relative positions of the chemical indicators are used to determine which type each chemical indicator is.

Each set or grouping of chemical indicators from the digital image are processed and their respective colors are compared to a table, library, mapping, index, etc. that associates a given color of chemical indicator to a given concentration level (block 614). As previously noted, the overall estimated concentration level can be based on averaging the individual concentration levels associated with each chemical indicator or by a voting mechanism. As part of processing the chemical indicators, certain individual chemical indicators can be determined to be valid or invalid (e.g., associated with an error) (block 616). For example, a chemical indicator may be determined to be invalid if it is determined that the needle with the chemical indicator has detached from the patient or otherwise has an issue that affects the color of the chemical indicator.

Further, in certain instances, the method 600 can include using the estimated concentration level(s) to update a database of historical concentration levels and analyze the concentration levels to determine trends and potential health risks (block 618). Further, in certain instances, the method 600 can include periodically estimating the remaining longevity or life of the wearable device (block 620). This can include analyzing information such as the ratio of valid to invalid chemical indicators, the service duration of the wearable device, the dispersion or spread of colors of the chemical indicators.

In certain instances, the method 600 includes determining whether the estimated analyte concentrations warrant treatment or action (block 622). If not, the method 600 can conclude (block 624). But if action is warranted, a notification (e.g., electronic message) can be sent to the patient's physician (block 626) along with the underlying data that caused the notification.

In certain instances, the method 600 is carried out by an application stored on and operated by a smart phone. In other instances, some or all steps of the method 600 can be carried out by a server or other computing system besides a smart phone that can access digital images of a wearable device and be programmed to determine estimated analyte concentration levels based on colors of chemical indicators shown in the digital image.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A system comprising:
a wearable device that includes:
a first set of needles sized for access to interstitial fluid;
a first type of chemical indicator associated with and positioned within each needle of the first set of needles;
a first set of membranes respectively housed within the first set of needles;
a second set of needles sized for access to interstitial fluid;
a second type of chemical indicator associated with and positioned within each needle of the second set of needles; and
a second set of membranes respectively houses within the second set of needles,
wherein the first set of membranes and the second set of membranes comprise an ion permeable polymeric matrix material.

2. The system of claim 1, wherein the first type of chemical indicator includes a first material that changes color to indicate a first concentration of a first chemical in the interstitial fluid, wherein the second type of chemical indicator includes a second material that changes color to indicate a second concentration of a second chemical in the interstitial fluid, wherein the first chemical and the second chemical are different.

3. The system of claim 2, wherein the wearable device further includes:
a third set of needles sized for access to interstitial fluid; and
a third type of chemical indicator associated with and positioned within each needle within the third set of needles,
wherein the third type of chemical indicator includes a third material that changes color to indicate a third concentration of a third chemical in the interstitial fluid, wherein the third chemical is different than the first chemical and the second chemical.

4. The system of claim 1, wherein the wearable device further includes color references positioned throughout the wearable device adjacent to the first type of chemical indicators and the second type of chemical indicators.

5. The system of claim 1, wherein the first type of chemical indicator is associated with sodium, wherein the second type of chemical indicator is associated with potassium.

6-7. (canceled)

8. The system of claim 1, wherein the wearable device further includes:
a moisture barrier comprising a window and arranged on the wearable device to seal the first type of chemical indicator in the first set of needles and to seal the second type of chemical indicator in the second set of needles.

9. The system of claim 8, wherein the wearable device further includes:
a protective film removably coupled to the moisture barrier.

10. The system of claim 1, wherein the wearable device is a patch attachable to a patient's skin.

11. The system of claim 1, further comprising:
a smart phone comprising an image sensor, wherein the smart phone is programmed to: determine a first concentration of a first analyte based, at least in part, on a first color of the first type of chemical indicator in an image from the image sensor.

12. A system comprising:
a wearable device that includes:
a first set of needles sized for access to interstitial fluid,
a first type of chemical indicator associated with and positioned within each needle of the first set of needles,
a second set of needles sized for access to interstitial fluid,
a second type of chemical indicator associated with and positioned within each needle of the second set of needles, and
respective diffusion membranes housed within the first set of needles and the second set of needles and comprising an ion permeable polymeric matrix material; and
a smart phone comprising an image sensor, wherein the smart phone is programmed to: (1) determine a first concentration of a first analyte based, at least in part, on a first color of the first type of chemical indicator, and (2) determine a second concentration of a second analyte based, at least in part, on a second color of the second type of chemical indicator.

13. The system of claim 12, wherein the first color and the second color are based on a digital image taken by the image sensor.

14. The system of claim 13, wherein the digital image includes color reference sections on the wearable device, wherein the first concentration and the second concentration are both further based on the color reference sections.

15. The system of claim 12, wherein the first analyte concentration is a sodium concentration, wherein the second analyte concentration is a potassium concentration.

16-20. (canceled)

21. The system of claim 12, wherein the ion permeable polymeric matrix material comprises a hydrogel.

22. The system of claim 21, wherein the first type of chemical indicator comprises lipophilic indicator dye.

23. The system of claim 22, wherein the lipophilic indicator dye comprises ionophores or fluorophores.

24. The system of claim 1, wherein the ion permeable polymeric matrix material comprises a hydrogel.

25. The system of claim 1, wherein the first type of chemical indicator comprises lipophilic indicator dye.

26. The system of claim 25, wherein the lipophilic indicator dye comprises ionophores or fluorophores.
